(19) United States
(12) Patent Application Publication
(10) Pub. No.: US 2009/0054389 A1
(43) Pub. Date: Feb. 26, 2009

(54) Topical Composition Comprising an Antibacterial Substance

(75) Inventor: Mette Rydahl Sonne, Brondby Strand (DK)

Correspondence Address: BIRCH STEWART KOLASCH & BIRCH, PO BOX 747, FALLS CHURCH, VA, 22040-0747, US

(73) Assignee: Leo Pharma A/S, Ballerup (DK)

(21) Appl. No.: 12/087,743

(22) PCT Filed: Feb. 1, 2007
(86) PCT No.: PCT/DK2007/000049

§ 371 Date: Aug. 12, 2008

Related U.S. Application Data
(60) Provisional application No. 60/764,345, filed on Feb. 2, 2006.

(51) Int. Cl. A61K 31/575; A61P 17/00; A61P 31/04

(52) U.S. Cl. 514/182

(57) ABSTRACT

A pharmaceutical composition for topical application comprises a fusidic acid derivative of general formula I as disclosed herein and one or more monoglycerides of a fatty acid. The composition may be used in the treatment of a disease or condition of the skin or mucosa, in particular skin infections.

### FIELD OF INVENTION

The present invention relates to a pharmaceutical composition for topical application comprising an antibacterial substance as an active component and the use of said composition in the treatment of diseases of skin or mucosa.

### BACKGROUND OF THE INVENTION

Fusidic acid belongs to the fusidanes which is a small family of naturally occurring antibiotics.

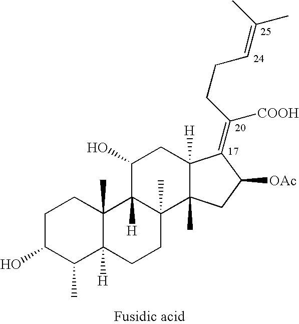

The fusidanes have in common a tetracyclic ring system with a unique chair-boat-chair conformation, which distinguishes them from steroids. Therefore, in spite of some structural similarity with steroids, namely a tetracyclic system, the fusidanes do not exert any hormonal activity. The fusidanes also have in common a carboxylic acid bearing side chain linked to the ring system at C-17 via a double bond and an acetate group linked at C-16. Fusidic acid, a fermentation product of Fusidium coccineum, is the most antibiotically active compound of the fusidanes and is the only fusidane used clinically in treatment of infectious diseases. Fusidic acid (Fucidin®) is used clinically for the treatment of severe staphylococcal infections, particularly in bone and joint infections, in both the acute and the intractable form of the disease (The Use of Antibiotics, 5^{th} Ed., A. Kucers and N. McK. Bennett (Eds.), Butterworth 1997, pp. 580-587, and references cited therein). Although fusidic acid is most commonly used against staphylococci, it is also used against several other gram-positive species. The clinical value of fusidic acid is also due to its efficient distribution in various tissues, low degree of toxicity and allergic reactions and the absence of cross-resistance with other clinically used antibiotics.

Fusidic acid is widely used in local therapy for a number of skin and eye infections caused by staphylococci. It may be administered as single therapy or given in combination with common antibiotics such as penicillins, erythromycins or clindamycin. It has also been used as an alternative to vancomycin for the control of Clostridium difficile. Compared to staphylococci, several other gram-positive cocci are often less susceptible to fusidic acid. As an example, streptococcal species are generally up to 100-fold less sensitive to fusidic acid than staphylococci [Kuchers et al. supra]. Other sensitive bacteria include gram-positive anaerobic cocci, such as Peptococcus and Peptostreptococcus spp., aerobic or anaerobic gram-positive bacteria, such as Corynebacterium diphtheriae, Clostridium tetani, Clostridium difficile and Clostridium perfringens. Gram-negative bacteria are resistant except for Neisseria spp. and Legionella pneumophila. The drug is highly potent against both intracellular and extracellular M. leprae.

EP 636 024 discloses a topical antimicrobial composition which includes glycerol monolaurate or glycerol monomyristate or a mixture of these monoglycerides as well as certain named antimicrobial compounds, e.g. fusidic acid. It is indicated in this publication that the monoglycerides and antimicrobial compounds exhibit a synergistic effect with respect to antimicrobial properties of the mixture.

### SUMMARY OF THE INVENTION

When preparing pharmaceutical compositions for topical application on the skin, an important consideration is that the therapeutically active component or components should be released from the vehicle and pass through the stratum corneum to the live layers of the skin, i.e. the epidermis and dermis. It is equally important to consider that the therapeutically active component or components should not immediately permeate through the skin into the systemic circulation where they might exert unwanted systemic side effects.

In the course of research leading to the present invention, the inventors have found that when a composition according to EP 636 024 containing fusidic acid in a monoglyceride-containing vehicle is applied on barrier impaired skin, a major proportion of the absorbed fusidic acid permeates through the skin and only a minor proportion is retained in the dermis and epidermis. In contrast, the present inventors have surprisingly found that when a fusidic acid derivative in a corresponding composition is applied on barrier impaired skin, a major proportion of the absorbed fusidic acid derivative is retained in the dermis and epidermis while only a minor proportion permeates through the skin and into the circulation.

Accordingly, the present invention relates to a pharmaceutical composition for topical application comprising a compound of general formula I

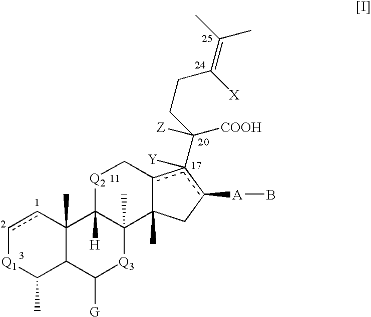

wherein X is halogen, trifluoromethyl, cyano, azido, alkyl, alkenyl or aryl, wherein said aryl may optionally be substituted by alkyl, alkenyl, halogen, azido, trifluoromethyl or cyano;

Y and Z are both hydrogen, or together with the C-17/C-20 bond form a double bond between C-17 and C-20, or together are methylene and form a cyclopropane ring in combination with C-17 and C-20;

A is a bond, O, S or S(O);

B is C₁₋₆ alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxyl, alkoxy and azido, or, if A is a bond, B may also be hydrogen; Q₁ and Q₂ are independently —CH₂—, —C(O)—, —(CHOH)—, —(CHOR)—, —(CHSH)—, —(NH)—, —(CHNH₂)— or —(CW)—, wherein R is C₁₋₆ alkyl and W is halogen, cyano, azido or trifluoromethyl;

Q₃ is —CH₂—, —C(O)— or —CHOH—;

G is H, OH or O—CO—CH₃;

two bonds in the pentacyclic ring being shown with full and dotted lines to indicate that either of the two bonds may be a double bond, in which case Y is absent and Z is hydrogen;

the bond between C-1 and C-2 being either a single or a double bond;

or a pharmaceutically acceptable salt or easily hydrolysable ester thereof, and a pharmaceutically acceptable vehicle comprising a monoglyceride of a C₈₋₁₈ fatty acid or a mixture of such monoglycerides.

Compounds of general formula I are disclosed in an earlier application by the present applicant published as WO 2005/007669, the contents of which are hereby incorporated by reference in their entirety.

In another aspect, the invention relates to a method of preventing or treating a dermal disease or condition of the skin or mucosa, the method comprising topically administering, to a patient in need thereof, an effective amount of the composition described above.

In a further aspect, the invention relates to the use of the composition described above for the manufacture of a medicament for the prevention or treatment of a disease or condition of the skin or mucosa.

### DETAILED DESCRIPTION OF THE INVENTION

### Definitions

In the present context, the term “alkyl” is intended to indicate a univalent radical derived from an alkane by removal of a hydrogen atom from any carbon atom, and includes the subclasses of primary, secondary and tertiary alkyl groups, including for example C₁-C₁₂ alkyl, such as C₁-C₈ alkyl, such as C₁-C₆ alkyl, such as C₁-C₄ alkyl, methyl, ethyl, n-propyl, isopropyl, n-butyl, isobutyl, sec-butyl, t-butyl, pentyl, hexyl, nonyl, dodecanyl, cyclopropyl, cyclopropylmethyl, cyclobutyl, cyclopentyl and cyclohexyl. Alkane refers to an acyclic or cyclic, branched or unbranched saturated hydrocarbon and therefore consisting entirely of hydrogen atoms and carbon atoms.

The term “alkenyl” is intended to indicate to a straight or branched acyclic hydrocarbon having one or more carbon-carbon double bonds of either E or Z stereochemistry where applicable. The term includes, for example, C₂-C₁₂ alkenyl, C₂-C₈ alkenyl, C₂-C₆ alkenyl, vinyl, allyl, 1-butenyl, 2-butenyl, and 2-methyl-2-propenyl.

The term “acyl” is intended to indicate a radical of the formula —CO—R, wherein R is alkyl as defined above, for example C₁-C₆ acyl.

The term “alkoxy” is intended to indicate a radical of the formula —OR, wherein R is alkyl as defined above, for example C₁-C₅ alkoxy, C₁-C₃ alkoxy, methoxy, n-propoxy, t-butoxy, and the like.

The term “halogen” indicates a member of the seventh main group of the periodical system, i.e. fluoro, chloro, bromo, and iodo; chloro, bromo and iodo being more useful in the present compounds.

The term “cycloalkylcarbonyl” is intended to indicate a radical of the formula —C(O)—R′, wherein R′ represents a cyclic alkyl as indicated above.

The term “aryl” is intended to indicate a cyclic, optionally a fused bicyclic, radical, wherein all ring atoms are carbon, and wherein the ring is aromatic, or in the case of a fused ring system, at least one ring is aromatic. Examples of aryl include phenyl, napthyl and tetralinyl.

The expression “easily hydrolysable esters” is used in this specification to denote alkanoyloxyalkyl, aralkanoyloxyalkyl, aroyloxyalkyl, for example acetoxymethyl, pivaloyloxymethyl, benzoyloxymethyl esters and the corresponding 1′-oxyethyl derivatives, or alkoxycarbonyloxyalkyl esters, for example methoxycarbonyloxymethyl esters and ethoxycarbonyloxymethyl esters, and the corresponding 1′-oxyethyl derivatives, or lactonyl esters, for example phthalidyl esters, or dialkylaminoalkyl esters, for example diethylaminoethyl esters. The expression “easily hydrolysable esters” includes in vivo hydrolysable esters of the compounds of the invention. Such esters may be prepared using methods known to a skilled person in the art, cf. GB patent No. 1 490 852 hereby incorporated by reference.

The term “monoglyceride” is intended to indicate a glyceryl monoester of a C₈₋₁₈ fatty acid, such as myristic, lauric, capric, caprylic, palmitoleic, palmitic, linoleic, linolenic or oleic acid. The term is also intended to include propylene glycol esters of monoglycerides such as propylene glycol caprylate or propylene glycol laurate.

The term “carbomer” is intended to indicate a polymer of acrylic acid crosslinked with polyalkenyl ethers of sugars or polyalcohols), such as allyl ethers of pentaerythritol, allyl ethers of sucrose. Examples of suitable carbomers include carbomer 910, carbomer 934, carbomer 934P, carbomer 940, carbomer 941, carbomer 971P, carbomer 974P, carbomer 980 or carbomer 981. The term “carbomer” may also include a copolymer of acrylic acid and long chain alkyl methacrylate cross-linked with allyl ethers of pentaerythritol, e.g. carbomer 1342, Carbopol® 1382, Carbopol® 2984 or Carbopol® 5984.

The term “poloxamer” is intended to indicate a polyoxyethylene-polyoxypropylene copolymer, e.g. poloxamer 124, poloxamer 188, poloxamer 237, poloxamer 338 or poloxamer 407.

The term “macrogol” is intended to indicate polyethylene glycol and is used synonymously herewith in the following.

The term “barrier impaired skin” is intended to indicate skin in which the outer layer, the stratum corneum, is not intact. Barrier impairment may be result of disease such as infection or eczema or may be artificially induced, e.g. by tape stripping as discussed in Example 3 below. Barrier impaired skin is more permeable to exogenous substances and has a higher transepidermal water loss than intact skin.

### Preferred Compounds of Formula I

Compounds of formula I are disclosed in WO 2005/007669 in which methods of making and using the compounds are also described in detail. The contents of WO 2005/07669 are incorporated by reference in their entirety.

Currently favoured compounds of formula I are those wherein Y and Z in the compound of formula I are both hydrogen and wherein the stereochemical configuration is S at both C-17 and C-20.

A in the compound of formula I may preferably be O or S(O).

X in the compound of formula I may preferably be fluoro, chloro, bromo, iodo, cyano, azido or trifluoromethyl.

Q₁ and Q₂ in the compound of formula I may independently represent —C(O)— or —(CHOH)—, or Q₁ in the compound of formula I may be CHF, CHCl, CHBr, CHI or CHN₃. In a particularly favoured embodiment, Q₁ or Q₂ in the compound of formula I or both Q₁ and Q₂ represent —(COH)— and the stereochemical configuration is a at both C-3 and C-11.

In currently favoured compounds of formula I, Q₁ and Q₂ may both be a —(CHOH)— group, or one of Q₁ or Q₂ may be —(CO)—, or Q₁ may be CHF, CHCl, CHBr, CHI or CHN3;

X may be chloro, bromo, iodo, trifluorometyl, azido or cyano;

Z and Y together with the C-17/C-20 bond form a double bond between C-17 and C-20;

A is oxygen;

B may be a C₁₋₄ alkyl group, optionally substituted with one or more substituents selected from the list consisting of azido, hydroxy, fluoro, chloro and bromo, or B is a C₁₋₄ acyl group or a benzoyl group, both optionally substituted with one or more halogen atoms, in particular chloro or bromo.

Thus, B in the compound of formula I may be ethyl, 2,2,2-trifluoroethyl, 2,2,2-trichloroethyl, 2-azidoethyl, 2-hydroxyethyl, propyl, tert.-butyl, isopropyl, 1,3-difluoro-isopropyl, acetyl, propionyl, chloroacetyl or trifluoroacetyl.

Specific examples of compounds of formula I are selected from the group consisting of

24-Trifluoromethyl fusidic acid sodium salt,
24-Trifluoromethyl fusidic acid pivaloyloxymethyl ester,
24-Chloro-fusidic acid,
24-Chloro-fusidic acid pivaloyloxymethyl ester,
24-Chloro-fusidic acid sodium salt,
24-Trifluoromethyl fusidic acid,
24-Bromo-fusidic acid acetoxymethyl ester,
24-Bromo-fusidic acid,
24-Bromo-fusidic acid sodium salt,
24-Bromo-fusidic acid pivaloyloxymethyl ester,
24-Bromo-16-deacetoxy-16β-thioacetyl-fusidic acid acetoxymethylester,
24-Bromo-16-deacetoxy-16β-isopropylthio-fusidic acid,
24-Bromo-16-deacetoxy-16β-isopropylsulfinyl-fusidic acid,
24-Bromo-16-deacetoxy-16β-thioacetyl-fusidic acid,
24-Bromo-17S,20S-dihydrofusidic acid,
24-Bromo-16-deacetoxy-16β-ethoxy-fusidic acid,
24-Bromo-16-deacetoxy-16β-ethoxy-fusidic acid acetoxymethyl ester,
24-Bromo-16-deacetoxy-16β-(2′,2′,2′-trifluoroethoxy)-fusidic acid acetoxymethyl ester,
24-Bromo-16-deacetoxy-16β-(2′,2′,2′-trifluoroethoxy)-fusidic acid,
24-Bromo-17S,20S-fusidic acid acetoxymethyl ester,
24-Bromo-17S,20S-methylene-fusidic acid acetoxymethyl ester,
24-Bromo-17S,20S-methylene-fusidic acid,
3-Deoxy-3β,24-dibromo-fusidic acid,
3α-Azido-24-bromo-3-deoxy-fusidic acid,
24-Iodo-fusidic acid,
24-Iodo-fusidic acid acetoxymethyl ester,
24-Iodo-fusidic acid pivaloyloxymethyl ester,
24-Phenyl-fusidic acid pivaloyloxymethylester,
24-Phenyl-fusidic acid,
24-(4-bromophenyl-fusidic acid pivaloyloxymethylester,
24-(4-bromophenyl)-fusidic acid,
24-(4-chlorophenyl)-fusidic acid pivaloyloxymethylester,
24-(4-chlorophenyl)-fusidic acid,
24-(3,5-difluorophenyl)-fusidic acid pivaloyloxymethylester,
24-(3,5-difluorophenyl)-fusidic acid, and
3-Deoxy-3β,24-Dibromo-fusidic acid acetoxymethyl ester.

### Preferred Compositions Comprising a Compound of Formula I

The monoglyceride or monoglycerides included in the present composition are preferably selected from glyceryl monomyristate, glyceryl monolaurate, glyceryl monocaprylate, glyceryl monocaprate, glyceryl monooleate, propylene glycol caprylate, propylene glycol laurate or propylene glycol monolaurate. In particular, the monoglyceride may be selected from glyceryl monomyristate or glyceryl monolaurate or a mixture thereof.

In a currently preferred embodiment, the monoglyceride comprises a mixture of glyceryl monomyristate and glyceryl monolaurate. In the composition, the ratio of glyceryl monomyristate to glyceryl monolaurate may favourably be in the range of about 1:5-5:1, in particular about 1:4-4:1, preferably about 1:3-3:1, such as about 1:2-2:1, e.g. about 1:2, about 1:1, about 2:1 or about 3:1.

In a currently favoured embodiment, the present composition also comprises a stabilising agent which serves to prevent phase separation of an aqueous and a lipid phase in the composition at elevated temperatures, e.g. temperatures of more than about 25° C. It has surprisingly been found that when a stabilising agent is included in the composition, an even higher proportion of the compound of formula I is retained in the dermis and epidermis on application of the composition on barrier impaired skin. Without wishing to be limited to any particular theory, it is currently believed that the stabilising agent has bioadhesive properties which might promote improved or prolonged contact to the skin resulting in the observed increased retention times of the compound of formula I in the dermis and epidermis.

The stabilising agent may be selected from a carbomer, poloxamer, cellulose derivative, polyvinylpyrrolidone or polyvinyl alcohol. Examples of suitable carbomers include carbomer 910, carbomer 934, carbomer 934P, carbomer 940, carbomer 941, carbomer 971P, carbomer 974P, carbomer 980, carbomer 981, carbomer 1342, Carbopol® 1382, Carbopol® 5984 or Carbopol® 2984. Examples of suitable cellulose derivatives include hydroxypropylcellulose (HPC), hydroxyethylcellulose (HEC), hydroxypropylmethylcellulose (HPMC), methyl cellulose (MC), or carboxymethylcellulose (CMC). Examples of suitable poloxamers include poloxamer 124, poloxamer 188, poloxamer 237, poloxamer 338 or poloxamer 407. Suitable polyvinylpyrrolidones include those which have a molecular weight (M_{w}) in the range of 7,000 to 1,500,000. Suitable polyvinyl alcohols include those which have a molecular weight (M_{w}) in the range of 30,000 to 200,000.

The present composition may also include an emulsifier. Suitable emulsifiers may be selected from polyethylene glycol stearate (e.g. PEG-100-stearate or macrogol 40 stearate), polyethylene glycol stearyl ether (e.g. Steareth 20), polyethylene glycol lauryl ether, polyethylene glycol cetostearyl ether, polysorbate, sorbitan oleate, cetyl alcohol or cetostearyl alcohol.

The present composition may also comprise other components used in topical formulations for dermal application, e.g. solvents (e.g. water or an alcohol or a mixture thereof), antioxidants (e.g. alpha-tocopherol or ascorbic acid), emollients (e.g. liquid paraffin, white soft paraffin, lanolin, isopropyl myristate, medium-chain triglycerides, hydrogenated castor oil, dimethicone), preservatives (e.g. diazolidinyl urea, methylparaben, propylparaben, ethylparaben, sorbic acid, or potassium sorbate), pH controlling agents (e.g. sodium hydroxide, hydrochloric acid or citric acid), skin soothing agents, skin healing agents and skin conditioning agents such as urea, glycerol, propylene glycol, sorbitol or bisabolol, cf. CFTA Cosmetic Ingredients Handbook, 2^{nd} Ed., 1992.

According to the invention, the present composition may comprise a compound of formula I formulated as a suspension or solution in a dermatologically acceptable vehicle in accordance with accepted pharmaceutical practice, e.g. as described in A. Williams, Transdermal and Topical Delivery Systems, Pharmaceutical Press, London and Chicago, 2003. Thus, the composition may be in the form of any topical formulation suitable for dermal application, including a cream, ointment, lotion, liniment, gel, spray, foam, suspension, or solution. The composition is preferably in the form of an aqueous gel or cream as these are generally more acceptable cosmetically.

The composition preferably has a pH at or near that of the surface of the skin, i.e. in the range of 4.0-7.0, more specifically 4.5-6.0.

As indicated above, it has surprisingly been found that a compound of formula I (referred to in the following as “the active component”) formulated in the present monoglyceride-containing vehicle has an improved skin penetration as opposed to a formulation of the compound in a conventional ointment or cream vehicle which does not contain any monoglyceride. On the other hand, it has also been found that a higher proportion of the active component is retained in the dermis and epidermis when the present composition is applied on barrier-impaired skin compared to a corresponding monoglyceride composition comprising fusidic acid.

Thus, in an experimental study of penetration of different radiolabelled compositions into barrier-impaired skin reported in Example 3 below, it has been found that when an active component is formulated in a monoglyceride vehicle according to the invention, about 50% of the total amount applied of the active component penetrates into the skin as opposed to about 25% of the total amount applied of the active component formulated in a conventional cream vehicle and about 10% of the total amount applied of the active component from a conventional ointment vehicle. On the other hand, about 10% and 15%, respectively, of the total amount applied of the active component permeate through the skin (i.e. potentially become available systemically) when formulated in two different monoglyceride compositions according to the invention, whereas about 35% of the fusidic acid formulated in a monoglyceride composition permeates through the skin. Due to the high degree of retention of the active compound of formula I in dermis and epidermis, the present monoglyceride composition containing a compound of formula I therefore appears to be uniquely suited for application on skin in case of a disease or condition involving barrier impairment of the skin, such as a skin infection or a disease involving a skin infection such as impetigo, acne, dermatitis, cellulitis, folloculitis or a superficial wound or injury, or a skin or mucosa infection caused by or involving the presence of a strain of Staphylococcus aureus, Streptococcus pyogenes, Corynebacterium xerosis, Staphylococcus epidermidis or Propionibacterium acnes.

It is currently expected that the amount of compound of formula I in the present composition may be in the range of from about 10 mg/g vehicle to about 40 mg/g vehicle, preferably from about 15 mg/g vehicle to about 30 mg/g vehicle, in particular from about 20 mg/g vehicle to about 25 mg/g vehicle.

According to the invention, the present composition may comprise one or more additional active components including, but not limited to, antimicrobial agents such as mupirocin, or anti-inflammatory agents such as corticosteroids, e.g. hydrocortisone, clobetasol, betamethasone, clobetasone, desoximethasone, diflucortolone, difloxasone, diflorasone, flumethasone, fluocinolone, fluticasone, fluprednidone, halcinomide, mometasone, triamcinolone, or pharmaceutically acceptable esters thereof, nicotinamide or derivatives thereof or calcineurin inhibitors such as tacrolimus or pimecrolimus.

The invention is described in further detail in the following examples which are not in any way intended to limit the scope of the invention as claimed.

### EXAMPLES

### Example 1

Monoglyceride composition A

| 24-bromo-fusidic acid | 20 | mg |
|---|---|---|
| Glyceryl monomyristate | 210 | mg |
| Glyceryl monolaurate | 70 | mg |
| Glycerol 85% | 60 | mg |
| Diazolidinyl urea | 5 | mg |
| Sodium citrate | 2.1 | mg |
| Citric acid monohydrate | 0.9 | mg |
| HCl | q.s. | |
| Water, purified | up to 1 | g |

Part of the aqueous phase was heated to about 70° C. and mixed with the melted oil phase (glyceryl monomyristate and glyceryl monolaurate) with homogenization. Part of the aqueous phase was mixed with citric acid monohydrate, sodium citrate, glycerol 85% and diazolidinyl urea and added to the cream blend with homogenization. The cream blend was cooled to room temperature with stirring. The pH was determined to be about 5. The 24-bromo-fusidic acid was added to the cream vehicle by means of a mortar and pestle.

For use in the skin penetration study described in Example 3 below, a labelled formulation was prepared by adding 11-³H 24-bromo-fusidic acid to the corresponding cooled cream vehicle by means of a mortar and pestle. 11-³H 24-bromo-fusidic acid was prepared by reduction of the corresponding 11-keto compound with NaBT₄ in methanol under mild basic conditions (pH adjusted to 8.0 with 1M sodium hydroxide) followed by chromatographic purification. The radioactivity of the composition was adjusted to 5 MBq/g.

Monoglyceride composition B

| 24-bromo-fusidic acid | 20 | mg |
|---|---|---|
| Glyceryl monomyristate | 140 | mg |
| Glyceryl monolaurate | 47 | mg |
| Glycerol 85% | 30 | mg |
| Diazolidinyl urea | 5 | mg |
| Carbomer 974P | 1.5 | mg |
| Butylhydroxyanisole | 25 | mcg |
| NaOH | q.s. | |
| Water, purified | up to 1 | g |

Part of the aqueous phase was heated to about 70° C. and mixed with the melted oil phase (glyceryl monolaurate, glyceryl monomyristate, butylhydroxyanisole) with homogenization. Part of the aqueous phase was mixed with glycerol 85%, diazolidinyl urea and carbomer 974P and added to the cream blend with stirring. The pH was adjusted to about 5. The cream blend was cooled to room temperature with stirring. The 24-bromo-fusidic acid was added to the cream vehicle by means of a mortar and pestle.

For use in the skin penetration study described in Example 3 below, a labelled formulation was prepared by adding 11-³H 24-bromo-fusidic acid to the corresponding cooled cream vehicle by means of a mortar and pestle. 11-³H 24-bromo-fusidic acid was prepared as described above for monoglyceride composition A. The radioactivity of the composition was adjusted to 5 MBq/g.

Monoglyceride composition C

| 24-bromo-fusidic acid | 20 | mg |
|---|---|---|
| Glyceryl monomyristate | 140 | mg |
| Glyceryl monolaurate | 47 | mg |
| Glycerol 85% | 30 | mg |
| Diazolidinyl urea | 5 | mg |
| Macrogol 40 stearate | 5 | mg |
| Carbomer 974P | 1.5 | mg |
| NaOH | q.s. | |
| Water, purified | up to 1 | g |

Part of the aqueous phase was heated to about 70° C. and mixed with the melted oil phase (glyceryl monolaurate, glyceryl monomyristate and macrogol 40 stearate) with homogenization. The cream blend was cooled to room temperature with stirring. Part of the aqueous phase was mixed with glycerol 85%, diazolidinyl urea and carbomer 974P and added to the cream blend with stirring. The pH was adjusted to about 5. The 24-bromo-fusidic acid was added to the cream vehicle by means of a mortar and pestle.

Monoglyceride composition D

| 24-Bromo-fusidic acid | 20 | mg |
|---|---|---|
| Glyceryl monomyristate | 50 | mg |
| Glyceryl monolaurate | 100 | mg |
| Glycerol 85% | 30 | mg |
| Diazolidinyl urea | 5 | mg |
| Macrogol 40 stearate | 5 | mg |
| Carbomer 974P | 1.5 | mg |
| NaOH | q.s. | |
| Water, purified | up to 1 | g |

Part of the aqueous phase was heated to about 70° C. and mixed with the melted oil phase (glyceryl monolaurate, glyceryl monomyristate and macrogol 40 stearate) with homogenization. The cream blend was cooled to room temperature with stirring. Part of the aqueous phase was mixed with glycerol 85%, diazolidinyl urea and carbomer 974P and added to the cream blend with stirring. The pH was adjusted to about 5. The 24-bromo-fusidic acid was added to the cream vehicle by means of a mortar and pestle.

Reference composition 1

| | | |
|---|---|---|
| 11-³H 24-Bromo-fusidic acid | 20 | mg |
| Cetyl alcohol | 111 | mg |
| Liquid paraffin | 111 | mg |
| Glycerol 85% | 111 | mg |
| Polysorbate 60 | 56 | mg |
| White soft paraffin | 56 | mg |
| Potassium sorbate | 2.7 | mg |
| Butylhydroxyanisole | 40 | mcg |
| Water, purified | up to 1 | g |

The aqueous phase (water, glycerol 85% and potassium sorbate) was heated to 70° C. and mixed with the melted oil phase (cetyl alcohol, liquid paraffin, polysorbate 60 and white soft paraffin) with homogenization. The pH was adjusted to about 5. The cream blend was cooled to room temperature while stirring.

The labelled formulation was made by adding 11-³H 24-bromo-fusidic acid, prepared as described above for monoglyceride composition A, to the corresponding cooled cream vehicle by means of a mortar and pestle. The radioactivity of the composition was adjusted to 5 MBq/g.

Reference composition 2

| | | |
|---|---|---|
| 11-³H 24-Bromo-fusidic acid (as sodium salt) | 20 | mg |
| Liquid paraffin | 140 | mg |
| Lanolin | 46 | mg |
| Cetyl alcohol | 4 | mg |
| α-tocopherol | 10 | mcg |
| White soft paraffin | up to 1 | g |

The ingredients were melted and mixed with homogenisation. The ointment was cooled with stirring. The 11-³H 24-bromo-fusidic acid (as sodium salt), prepared as described above for monoglyceride composition A, was added to the ointment vehicle by means of a mortar and pestle. The radioactivity of the formulation was adjusted to 5 MBq/g.

Reference composition 3

| 11-³H Fusidic acid (as hemihydrate) | 20 | mg |
|---|---|---|
| Glyceryl monomyristate | 210 | mg |
| Glyceryl monolaurate | 70 | mg |
| Glycerol 85% | 60 | mg |
| Diazolidinyl urea | 5 | mg |
| Sodium citrate | 2.1 | mg |
| Citric acid monohydrate | 0.9 | mg |
| HCl | q.s. | |
| Water, purified | up to 1 | g |

Part of the aqueous phase was heated to about 70° C. and mixed with the melted oil phase (glyceryl monomyristate and glyceryl monolaurate) with homogenization. Part of the aqueous phase was mixed with citric acid monohydrate, sodium citrate, glycerol 85% and diazolidinyl urea and added to the cream blend with homogenization. The cream blend was cooled to room temperature with stirring.

The labelled formulation was made by adding 11-³H 24-fusidic acid (as hemihydrate) to the corresponding cooled cream vehicle by means of a mortar and pestle. 11-³H fusidic acid was prepared by reduction of the corresponding 11-keto compound with NaBT₄ in methanol under mild basic conditions (pH adjusted to 8.0 with 1M sodium hydroxide) followed by chromatographic purification. The radioactivity of the composition was adjusted to 5 MBq/g.

### Example 2

### Monoglyceride Vehicle Compositions

Vehicle composition I

| Glyceryl monomyristate | 200 | mg |
|---|---|---|
| Glyceryl monolaurate | 200 | mg |
| Glycerol 85% | 60 | mg |
| Diazolidinyl urea | 5 | mg |
| Sodium citrate | 2.1 | mg |
| Citric acid monohydrate | 0.9 | mg |
| HCl | q.s. | |
| Water, purified | up to 1 | g |

Part of the aqueous phase, including glycerol 85%, was heated to about 70° C. and mixed with the melted oil phase (glyceryl monolaurate and glyceryl monomyristate) with homogenization. Part of the aqueous phase was mixed with diazolidinyl urea, sodium citrate and citric acid monohydrate, the pH was adjusted to about 5, and the mixture was added to the cream blend at about 55-65° C. with homogenization. The cream blend was cooled to room temperature with stirring.

Vehicle composition II

| Glyceryl monomyristate | 100 | mg |
|---|---|---|
| Glyceryl monolaurate | 200 | mg |
| Glycerol 85% | 60 | mg |
| Diazolidinyl urea | 5 | mg |
| Sodium citrate | 2.1 | mg |
| Citric acid monohydrate | 0.9 | mg |
| HCl | q.s. | |
| Water, purified | up to 1 | g |

Part of the aqueous phase, including glycerol 85%, was heated to about 70° C. and mixed with the melted oil phase (glyceryl monolaurate and glyceryl monomyristate) with homogenization. Part of the aqueous phase was mixed with diazolidinyl urea, sodium citrate and citric acid monohydrate, the pH was adjusted to about 5, and the mixture was added to the cream blend at about 55-65° C. with homogenization. The cream blend was cooled to room temperature with stirring.

Vehicle composition III

| Glyceryl monomyristate | 140 | mg |
|---|---|---|
| Paraffin, liquid | 50 | mg |
| Glyceryl monolaurate | 47 | mg |
| Glycerol 85% | 30 | mg |
| Macrogol 40 stearate | 5 | mg |
| Diazolidinyl urea | 5 | mg |
| Carbomer 974P | 1.5 | mg |
| NaOH | q.s | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea and carbomer 974P and added to the cream blend at about 30° C. with a whisk. The pH was adjusted to about 5.

Vehicle composition IV

| Glyceryl monomyristate | 140 | mg |
|---|---|---|
| Glyceryl monolaurate | 47 | mg |
| Triglyceride, medium chain | 30 | mg |
| Glycerol 85% | 30 | mg |
| Macrogol 40 stearate | 5 | mg |
| Diazolidinyl urea | 5 | mg |
| Carbomer 974P | 1.5 | mg |
| NaOH | q.s | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea and carbomer 974P and added to the cream blend at about 30° C. with a whisk. The pH was adjusted to about 5.

Vehicle composition V

| Glyceryl monomyristate | 140 | mg |
|---|---|---|
| Glyceryl monolaurate | 47 | mg |
| Glycerol 85% | 30 | mg |
| Macrogol 40 stearate | 5 | mg |
| Diazolidinyl urea | 5 | mg |
| Carbomer 974P | 1.5 | mg |
| Dimethicone | 2 | mg |
| NaOH | q.s | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea and carbomer 974P and added to the cream blend at about 30° C. with a whisk. The pH was adjusted to about 5.

Vehicle composition VI

| Glyceryl monomyristate | 210 | mg |
|---|---|---|
| Glyceryl monolaurate | 70 | mg |
| Glycerol 85% | 60 | mg |
| Steareth 20 | 10 | mg |
| Diazolidinyl urea | 5 | mg |
| Sodium citrate | 2.1 | mg |
| Citric acid monohydrate | 0.9 | mg |
| HCl | q.s | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea, citric acid monohydrate, sodium citrate, HCl (to pH 5) and added to the cream blend at about 50-60° C. with stirring. The resulting cream was cooled with stirring.

Vehicle composition VII

| Glyceryl monomyristate | 210 | mg |
|---|---|---|
| Glyceryl monolaurate | 70 | mg |
| Glycerol 85% | 60 | mg |
| PEG-100-stearate | 20 | mg |
| Diazolidinyl urea | 5 | mg |
| Sodium citrate | 2.1 | mg |
| Citric acid monohydrate | 0.9 | mg |
| HCl | q.s | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea, citric acid monohydrate, sodium citrate, HCl (to pH 5) and added to the cream blend at about 50-60° C. with stirring. The resulting cream was cooled with stirring.

Vehicle composition VIII

| | | |
|---|---|---|
| Glyceryl monomyristate | 200 | mg |
| Glycerol 85% | 60 | mg |
| Diazolidinyl urea | 5 | mg |
| Sodium citrate | 2.1 | mg |
| Citric acid monohydrate | 0.9 | mg |
| Water, purified | up to 1 | g |

Part of the aqueous phase and glycerol 85% was heated to about 70° C. and mixed with the melted oil phase with homogenisation. Part of the aqueous phase was mixed with diazolidinyl urea, citric acid monohydrate, sodium citrate and added to the cream blend with stirring and cooling.

Vehicle composition IX

| | | |
|---|---|---|
| Glyceryl monolaurate | 200 | mg |
| Glycerol 85% | 60 | mg |
| Diazolidinyl urea | 5 | mg |
| Sodium citrate | 2.1 | mg |
| Citric acid monohydrate | 0.9 | mg |
| Water, purified | up to 1 | g |

Part of the aqueous phase and glycerol 85% was heated to about 70° C. and mixed with the melted oil phase with homogenisation. Part of the aqueous phase was mixed with diazolidinyl urea, citric acid monohydrate, sodium citrate and added to the cream blend with stirring and cooling.

Vehicle composition X

| Glyceryl myristate | 140 | mg |
|---|---|---|
| Poloxamer 407 | 50 | mg |
| Glyceryl laurate | 47 | mg |
| Glycerol 85% | 30 | mg |
| Diazolidinyl urea | 5 | mg |
| Macrogol 40 stearate | 5 | mg |
| HCl | q.s. | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea, poloxamer 407 and added to the cream blend at about 30° C. with a whisk. The pH was adjusted to about 5.

Vehicle composition XI

| Glyceryl laurate | 100 | mg |
|---|---|---|
| Glyceryl myristate | 50 | mg |
| Poloxamer 407 | 50 | mg |
| Glycerol 85% | 30 | mg |
| Diazolidinyl urea | 5 | mg |
| Macrogol 40 stearate | 5 | mg |
| HCl | q.s. | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea, poloxamer 407 and added to the cream blend at about 30° C. with a whisk. The pH was adjusted to about 5.

Vehicle composition XII

| Glyceryl myristate | 100 | mg |
|---|---|---|
| Glyceryl laurate | 50 | mg |
| Glycerol 85% | 30 | mg |
| Diazolidinyl urea | 5 | mg |
| Macrogol 40 stearate | 5 | mg |
| Sodium carboxymethylcellulose | 2 | mg |
| HCl | q.s. | |
| Water, purified | up to 1 | g |

Part of the aqueous phase was heated to about 70° C. and mixed with the melted oil phase with homogenisation. Part of the aqueous phase was mixed with glycerol 85%, diazolidinyl urea, sodium carboxymethylcellulose and added to the cream blend at about 30° C. with a whisk. The pH was adjusted to about 5.

To make pharmaceutical compositions according to the invention, an active ingredient such as 24-bromo-fusidic acid (20 mg) may be added to the vehicle compositions I-XII described above with homogenization, followed by adjusting the pH to about 5 before the compositions are cooled to room temperature. Alternatively, the active ingredient may be added to the cooled cream vehicle by means of a mortar and pestle.

### Example 3

In vitro Skin Penetration Study

Full thickness skin from pig ears was used in the study. The ears were kept frozen at −18° C. before use. On the day prior to the experiment the ears were placed in a refrigerator (5±3° C.) for slow defrosting. On the day of the experiment, the hairs were removed using a veterinary hair trimmer. The skin was cleaned for subcutaneous fat using a scalpel and two pieces of skin were cut from each ear and mounted on Franz diffusion cells in a balanced order.

For barrier impairment, the skin was tape stripped 25 times using D-Squame® tape (diameter 22 mm, CuDerm Corp., Dallas, Tex., USA). Each tape was applied to the test area using standard pressure for 5 seconds and removed from the test area in one gentle continuous move. The tearing-off direction was varied for each repeated strip.

Static Franz-type diffusion cells with an available diffusion area of 3.14 cm² and receptor volumes ranging from 8.6 to 11.1 ml were used in substantially the manner described by T. J. Franz, “The finite dose technique as a valid in vitro model for the study of percutaneous absorption in man”, in Current Problems in Dermatology, 1978, J. W. H. Mall (Ed.), Karger, Basel, pp. 58-68. The specific volume was measured and registered for each cell. A magnetic bar was placed in the receptor compartment of each cell. After mounting the skin, physiological saline (35° C.) was filled into each receptor chamber for hydration of the skin. The cells were placed in a thermally controlled water bath which was placed on a magnetic stirrer set at 400 rpm. The circulating water in the water baths was kept at 35±1° C. resulting in a temperature of about 32° C. on the skin surface. After one hour the saline was replaced by receptor medium, 0.04 M isotonic phosphate buffer, pH 7.4 (35° C.). Sink conditions were maintained at all times during the period of the study, i.e. the concentration of the active compounds in the receptor medium was below 10% of the solubility of the compounds in the medium.

Fusidic acid and 24-bromo-fusidic acid, respectively, was labelled with ³H as described in Example 1 before being added to each of the test compositions (monoglyceride compositions A and B, and reference compositions 1, 2 and 3). The in vitro skin permeation of each test composition was tested in 6 replicates (i.e. n=6). Duplicate samples of each of the radioactive compositions were analysed and used as references to test the radioactivity of the compositions. Each test composition was applied to the skin membrane at 0 hours in an intended dose of 4 mg/cm². A glass spatula was used for the application, and the residual amount of the composition was determined so as to give the amount of the composition actually applied on the skin.

The skin penetration experiment was allowed to proceed for 21 hours. Samples were then collected from the following compartments:

(1) Excess composition on the inside of the donor part of the diffusion cell and excess composition on the skin surface was collected with a cotton swab and tape stripping twice with Transpore® tape (available from 3M Healthcare, Minneapolis, USA).
(2) Viable epidermis was separated after 3 minutes of exposure to 60° C. at high humidity.
(3) The dermis was cut into smaller pieces and distributed to several scintillation vials.
(4) The skin surrounding the test area was cut into smaller pieces and analysed separately to gain information on lateral skin diffusion.
(5) Samples of the receptor fluid (1.0 ml, n=2) were analysed.

The samples were tested for their content of radioactivity using a liquid scintillation counter (Packard LSA 2100). The radioactivity in the samples was expressed as disintegration per minute (DPM), and DPM values were calculated as ng of the active compounds. The distribution of the active compound in the skin samples after 21 hours, i.e. the amount expressed as nmol/cm² and % of the applied dose present in viable epidermis, dermis and receptor fluid, was determined. Furthermore, the amount of active compounds in the excess composition in the donor part of the diffusion cell and on skin surfaces as well as the amount of active compounds in the skin surrounding test area (i.e. lateral diffusion) was determined.

The results appear from Table 1 below which shows the total mass balance after topical application of monoglyceride compositions A and B as well as reference compositions 1, 2 and 3.

TABLE 1
Mass balance after topical application to barrier-impaired skin in percentage
of the applied amount (mean values ± SD, n = 6)

| Test | Skin | Donor | | | Receptor | Lateral | |
|---|---|---|---|---|---|---|---|
| compound | surface | cell | Epidermis | Dermis | fluid | diffusion | Total |
| Ref. | 59 ± 8 | 9 ± 3 | 14 ± 2 | 4 ± 3 | 5 ± 4 | 3 ± 1 | 95 ± 1 |
| compos. 1 | | | | | | | |
| Ref. | 77 ± 9 | 10 ± 5 | 4 ± 1 | 3 ± 1 | 4 ± 2 | 3 ± 2 | 103 ± 8 |
| compos. 2 | | | | | | | |
| MG | 41 ± 21 | 7 ± 6 | 19 ± 4 | 17 ± 10 | 17 ± 10 | 3 ± 1 | 104 ± 3 |
| compos. A | | | | | | | |
| MG | 41 ± 20 | 4 ± 2 | 24 ± 14 | 15 ± 13 | 10 ± 4 | 3 ± 1 | 97 ± 5 |
| compos. B | | | | | | | |
| Ref. | 39 ± 10 | 4 ± 2 | 11 ± 1 | 9 ± 1 | 36 ± 10 | 2 ± 2 | 101 ± 5 |
| compos. 3 | | | | | | | |

Based on these results, it may be concluded that the skin penetration of 11-3 H 24-bromo-fusidic acid from monoglyceride compositions A and B is significantly higher than when 11-³H 24-bromo-fusidic acid is formulated in conventional cream and ointment vehicles (reference compositions 1 and 2, respectively). Furthermore, the degree of retention of 11-³H 24-bromo-fusidic acid in dermis and epidermis is significantly higher than that of 11-³H fusidic acid in a monoglyceride composition.

1. A pharmaceutical composition for topical application comprising a compound of general formula I 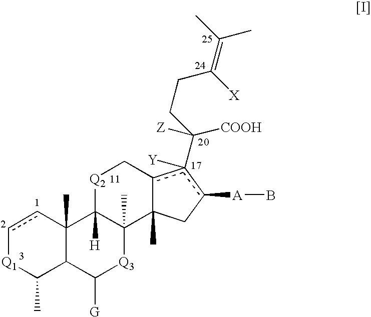
wherein X is halogen, trifluoromethyl, cyano, azido, alkyl, alkenyl or aryl, wherein said aryl may optionally be substituted by alkyl, alkenyl, halogen, azido, trifluoromethyl or cyano;
Y and Z are both hydrogen, or together with the C-17/C-20 bond form a double bond between C-17 and C-20, or together are methylene and form a cyclopropane ring in combination with C-17 and C-20;
A is a bond, O, S or S(O);
B is C₁₋₆alkyl, C₂₋₆ alkenyl, C₁₋₆ acyl, C₃₋₇ cycloalkylcarbonyl or benzoyl, all of which are optionally substituted with one or more substituents selected from the group consisting of halogen, hydroxyl, alkoxy and azido, or, if A is a bond, B may also be hydrogen;
Q₁ and Q₂ are independently —CH₂—, —C(O)—, —(CHOH)—, —(CHOR)—, —(CHSH)—, —(NH)—,
—(CHNH₂)— or —(CW)—, wherein R is C₁₋₆ alkyl and W is halogen, cyano, azido or trifluoromethyl;
Q₃ is —CH₂—, —C(O)— or —CHOH—;
G is H, OH or O—CO—CH₃;
two bonds in the pentacyclic ring being shown with full and dotted lines to indicate that either of the two bonds may be a double bond, in which case Y is absent and Z is hydrogen;
the bond between C-1 and C-2 being either a single or a double bond;
or a pharmaceutically acceptable salt or easily hydrolysable ester thereof, and a pharmaceutically acceptable vehicle comprising a monoglyceride of a C₈₋₁₈fatty acid or a mixture of such monoglycerides.

2. A composition according to claim 1, wherein the monoglyceride is glyceryl monomyristate, glyceryl monolaurate, glyceryl monocaprylate, glyceryl monocaprate, glyceryl monooleate, propylene glycol caprylate or propylene glycol laurate.

3. A composition according to claim 2, wherein the monoglyceride is glyceryl monomyristate or glyceryl monolaurate or a mixture thereof.

4. A composition according to claim 3, wherein the monoglyceride is a mixture of glyceryl monomyristate and glyceryl monolaurate.

5. A composition according to claim 4, wherein the ratio of glyceryl monomyristate to glyceryl monolaurate is in the range of about 1:5-5:1, in particular about 1:4-4:1, preferably about 1:3-3:1, such as about 1:2-2:1, e.g. about 1:2, about 1:1, about 2:1 or about 3:1.

6. A composition according to claim 1 further comprising a stabilising agent.

7. A composition according to claim 6, wherein the stabilising agent is a carbomer, poloxamer, cellulose derivative, polyvinylpyrrolidone or polyvinyl alcohol.

8. A composition according to claim 7, wherein the carbomer is carbomer 910, carbomer 934, carbomer 934P, carbomer 940, carbomer 941, carbomer 971P, carbomer 974P, carbomer 980, carbomer 981, carbomer 1342, Carbopol® 1382, Carbopol® 5984 or Carbopol® 2984.

9. A composition according to claim 7, wherein the cellulose derivative is hydroxypropylcellulose (HPC), hydroxyethylcellulose (HEC), hydroxypropylmethylcellulose (HPMC), methyl cellulose (MC), or carboxymethylcellulose (CMC).

10. A composition according to claim 7, wherein the poloxamer is poloxamer 124, poloxamer 188, poloxamer 237, poloxamer 338 or poloxamer 407.

11. A composition according to claim 7, wherein the polyvinylpyrrolidone has a molecular weight (Mw) in the range of 7,000 to 1,500,000.

12. A composition according to claim 7, wherein the polyvinyl alcohol has a molecular weight (M_{w}) in the range of 30,000 to 200,000.

13. A composition according to claim 1 further comprising an emulsifier.

14. A composition according to claim 13, wherein the emulsifier is selected from the group consisting of polyethylene glycol stearate, polyethylene glycol stearyl ether, polyethylene glycol lauryl ether, polyethylene glycol cetostearyl ether, polysorbate, sorbitan oleate, cetyl alcohol or cetostearyl alcohol.

15. A composition according to claim 1, wherein Y and Z in the compound of formula I are both hydrogen and wherein the stereochemical configuration is S at both C-17 and C-20.

16. A composition according to claim 1, wherein A in the compound of formula I is O or S(O).

17. A composition according to claim 1, wherein X in the compound of formula I is fluoro, chloro, bromo, iodo, cyano, azido or trifluoromethyl.

18. A composition according to claim 1, wherein Q₁ and Q₂ in the compound of formula I independently represent —C(O)— or —(CHOH)—.

19. A composition according to claim 1, wherein Q₁ in the compound of formula I is CHF, CHCl, CHBr, CHI or CHN₃.

20. A composition according to claim 1, wherein, in the compound of formula I, Q₁ and Q₂ are both a
—(CHOH)— group, or one of Q₁ or Q₂ is —(CO)—, or Q₁ is CHF, CHCl, CHBr, CHI or CHN3;
X is chloro, bromo, iodo, trifluorometyl, azido or cyano;
Z and Y together with the C-17/C-20 bond form a double bond between C-17 and C-20;
A is oxygen;
B is a C₁₋₄ alkyl group, optionally substituted with one or more substituents selected from the list consisting of azido, hydroxy, fluoro, chloro and bromo, or B is a C₁₋₄ acyl group or a benzoyl group, both optionally substituted with one or more halogen atoms.

21. A composition according to claim 20, wherein the halogen atoms with which B is optionally substituted are chloro or bromo.

22. A composition according to claims 20, wherein B in the compound of formula I is ethyl, 2,2,2-trifluoroethyl, 2,2,2-trichloroethyl, 2-azidoethyl, 2-hydroxyethyl, propyl, tert.-butyl, isopropyl, 1,3-difluoro-isopropyl, acetyl, propionyl, chloroacetyl or trifluoroacetyl.

23. A composition according to claim 1, wherein Q₁ or Q₂ in the compound of formula I or both Q₁ and Q₂ represent —(COH)— and the stereochemical configuration is a at both C-3 and C-11.

24. A composition according to claim 1, wherein the compound of formula I is selected from the group consisting of
24-Trifluoromethyl fusidic acid sodium salt,
24-Trifluoromethyl fusidic acid pivaloyloxymethyl ester,
24-Chloro-fusidic acid,
24-Chloro-fusidic acid pivaloyloxymethyl ester,
24-Chloro-fusidic acid sodium salt,
24-Trifluoromethyl fusidic acid,
24-Bromo-fusidic acid acetoxymethyl ester,
24-Bromo-fusidic acid,
24-Bromo-fuisidic acid sodium salt,
24-Bromo-fusidic acid pivaloyloxymethyl ester,
24-Bromo-16-deacetoxy-16β-thioacetyl-fusidic acid acetoxymethylester,
24-Bromo-16-deacetoxy-16β-isopropylthio-fusidic acid,
24-Bromo-16-deacetoxy-16β-isopropylsulfinyl-fusidic acid,
24-Bromo-16-deacetoxy-16β-thioacetyl-fusidic acid,
24-Bromo-17S,20S-dihydrofusidic acid,
24-Bromo-16-deacetoxy-16β-ethoxy-fusidic acid,
24-Bromo-16-deacetoxy-16β-ethoxy-fusidic acid acetoxymethyl ester,
24-Bromo-16-deacetoxy-16β-(2′,2′,2′-trifluoroethoxy)-fusidic acid acetoxymethyl ester,
24-Bromo-16-deacetoxy-16β-(2′,2′,2′-trifluoroethoxy)-fusidic acid,
24-Bromo-17S,20S-fusidic acid acetoxymethyl ester,
24-Bromo-17S,20S-methylene-fusidic acid acetoxymethyl ester,
24-Bromo-17S,20S-methylene-fusidic acid,
3-Deoxy-3α,24-dibromo-fusidic acid,
3α-Azido-24-bromo-3-deoxy-fusidic acid,
24-Iodo-fusidic acid,
24-Iodo-fusidic acid acetoxymethyl ester,
24-Iodo-fusidic acid pivaloyloxymethyl ester,
24-Phenyl-fusidic acid pivaloyloxymethylester,
24-Phenyl-fuisidic acid,
24-(4-bromophenyl)-fusidic acid pivaloyloxymethylester,
24-(4-bromophenyl)-fuisidic acid,
24-(4-chlorophenyl)-fusidic acid pivaloyloxymethylester,
24-(4-chlorophenyl)-fusidic acid,
24-(3,5-difluorophenyl)-fusidic acid pivaloyloxymethylester,
24-(3,5-difluorophenyl)-fusidic acid, and
3-Deoxy-3β,24-Dibromo-fusidic acid acetoxymethyl ester.

25. A composition according to claim 1 which is a gel or cream.

26. A composition according to claim 1 which has a pH in the range of 4.0-7.0, in particular about 4.5-6.0.

27. A composition according to claim 1, wherein the amount of compound of formula I is in the range of from about 10 mg/g vehicle to about 40 mg/g vehicle, preferably from about 15 mg/g vehicle to about 30 mg/g vehicle, in particular from about 20 mg/g vehicle to about 25 mg/g vehicle.

28. Use of a composition according to claim 1 for the manufacture of a medicament for the prevention or treatment of a disease or condition of the skin or mucosa.

29. The use of claim 28, wherein the disease or condition is a skin infection or a disease involving a skin infection, e.g. impetigo, acne, dermatitis, cellulitis, folloculitis or a superficial wound or injury, or a skin or mucosa infection caused by or involving the presence of a strain of Staphylococcus aureus, Streptococcus pyogenes, Corynebacterium xerosis, Staphylococcus epidermis or Propionibacterium acnes.

30. A method of preventing or treating a dermal disease or condition of the skin or mucosa, the method comprising topically administering, to a patient in need thereof, an effective amount of a composition according to claim 1.

31. The method of claim 30, wherein the disease or condition is a skin infection or a disease involving a skin infection, e.g. impetigo, acne, dermatitis, cellulitis, folloculitis or a superficial wound or injury, or a skin or mucosa infection caused by or involving the presence of a strain of Staphylococcus aureus, Streptococcus pyogenes, Corynebacterium xerosis, Staphylococcus epidermis or Propionibacterium acnes.
